(19) United States
(12) Patent Application Publication
(10) Pub. No.: US 2007/0226009 A1
(43) Pub. Date: Sep. 27, 2007

(54) METHODS AND SYSTEMS FOR PRESCRIPTION REVIEW TO IDENTIFY SUBSTITUTIONS

(76) Inventor: H. Don Hicks, Roswell, GA (US)

Correspondence Address: NEEDLE & ROSENBERG, P.C., SUITE 1000, 999 PEACHTREE STREET, ATLANTA, GA, 30309-3915, US

(21) Appl. No.: 11/691,228

(22) Filed: Mar. 26, 2007

Related U.S. Application Data
(60) Provisional application No. 60/785,874, filed on Mar. 24, 2006.

(51) Int. Cl. G06Q 10/00; A61B 5/00

(52) U.S. Cl. 705/2; 600/300

(57) ABSTRACT

Provided are methods and systems for prescription review and substitution. Embodiments can allow for selecting a more cost favorable pharmaceutical regime.

### CROSS REFERENCE TO RELATED APPLICATION

This application claims priority to U.S. Provisional Application No. 60/785,874 filed Mar. 24, 2006, herein incorporated by reference in its entirety.

### BACKGROUND

Prescriptions for various pharmaceuticals are written many times a day. Individuals (patients) will often have one or more prescriptions given to them by their treating physicians. Pharmaceuticals can have varying delivery regimes, dosages, and formulations. There can be several different pharmaceuticals that can be used to treat the same condition. The patient can be faced with a myriad of options including such considerations as insurance coverage or the use of generics.

### SUMMARY

Embodiments of the methods and systems provided can facilitate the review and comparison of a patient's current pharmaceutical prescription (also simply called “prescription” herein) with optional substitutes or alternative prescriptions. Exemplary methods can be implemented in whole or in part in a computing environment, including but not limited to the World Wide Web, also called the internet Embodiments can comprise methods implemented in a computing environment whereby a patient or someone on the patient's behalf can provide current prescription information for the patient and health insurance information for the patient, alternative prescriptions can be generated and provided to the patient, alternative prescriptions can be selected by the patient, a new prescription order can be generated containing both the currently prescribed medications and alternative prescriptions and the prescription order can be sent to a prescribing physician for approval as a new prescription to be filled.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

The accompanying drawings, which are incorporated in and constitute a part of this specification, illustrate embodiments and together with the description, serve to explain the principles of the methods and systems:

FIG. 1 is a flow diagram illustrating an exemplary implementation of the disclosed method;

FIG. 2 is a flow diagram illustrating an exemplary implementation of the disclosed method;

FIG. 3 is an illustrative transaction environment;

FIG. 4 is an exemplary prescription generated by the methods and systems disclosed;

FIG. 5 illustrates relationships generated through use of the disclosed methods and systems, and the synergy thus created; and

FIG. 6 is an exemplary operating environment.

### DETAILED DESCRIPTION

Before the present methods and systems are disclosed and described, it is to be understood that the methods and systems are not limited to specific synthetic methods, specific components and, as such may vary. It is also to be understood that the terminology used herein is for the purpose of describing particular embodiments only and is not intended to be limiting.

As used in the specification and the appended claims, the singular forms “a”, “an” and “the” include plural referents unless the context clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about”, it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not. “Alternative” means possible options, one or more of which may be satisfactory.

The Health Insurance Portability and Protection Act of 1996 [HIPPA] requires medical professionals to protect patients' privacy by requiring safeguards to be put in place by medical professionals to ensure confidentiality. Most professionals are required to comply with HIPPA. The regulations promulgated by the Department of Health and Human Services ensure a national floor of privacy protections for patients by limiting the ways that health plans, pharmacies, hospitals and other covered entities can use patients' personal medical information. The regulations protect medical records and other individually identifiable health information, whether it is on paper, in computers or communicated orally.

HIPPA sets limits on how health plans and covered providers may use individually identifiable health information. To promote the best quality care for patients, the rule does not restrict the ability of doctors, nurses and other providers to share information needed to treat their patients. HIPPA permits doctors and other covered entities to communicate freely with patients about treatment options and other health-related information, including disease-management programs.

Under HIPPA, patients can request that their doctors, health plans and other covered entities take reasonable steps to ensure that their communications with the patient are confidential. For example, a patient could ask a doctor to call his or her office rather than home, and the doctor's office should comply with that request if it can be reasonably accommodated.

HIPPA requires health plans, pharmacies, doctors and other covered entities to establish policies and procedures to protect the confidentiality of protected health information about their patients. These requirements are flexible and scalable to allow different covered entities to implement them as appropriate for their businesses or practices. Covered entities must provide all the protections for patients cited above, such as providing a notice of their privacy practices and limiting the use and disclosure of information as required under the rule. In addition, covered entities must take some additional steps to protect patient privacy.

The present methods and systems are configured to be in full compliance with the aforementioned HIPPA requirements. The present methods and systems may be understood more readily by reference to the following detailed description of preferred embodiments and the Examples included therein and to the Figures and their previous and following description.

Patients obtain prescriptions from their treating physicians in order to treat or prevent various diseases or ailments. Substitutions can be allowed, but generally, to change a prescription or dosage and frequency, a new prescription order must be approved by the treating physician.

Often, more than one pharmaceutical can have the same desired medical effect. Often, the same pharmaceutical (or medication) is available in different dosages where the desired medical effect can be gained, using one or more different dosage/frequency of administration combinations. For instance, for certain medications, a 500 mg dosage taken twice a day could be substituted with a 250 mg dosage taken 4 times a day. These differing medications and/or dosages may have a resulting total cost difference for the patient. Optionally, insurance benefits may influence the total cost to the patient as well, in that insurance policies may pay more or less for certain medications or dosages of the same medication.

Altering or changing a prescription can require the approval of the treating physician. Such approval can be obtained by a third party with the consent of the patient, or by the patient.

In an embodiment, provided is a method for identifying an alternative prescription comprising receiving a patient's current prescription information, identifying suitable alternative prescriptions and said alternative's pricing information, reviewing with the patient said alternatives whereby the patient selects one or more alternative prescriptions, and contacting the patient's prescribing physician, with the consent of the patient, to obtain physician approval for alternative prescription(s).

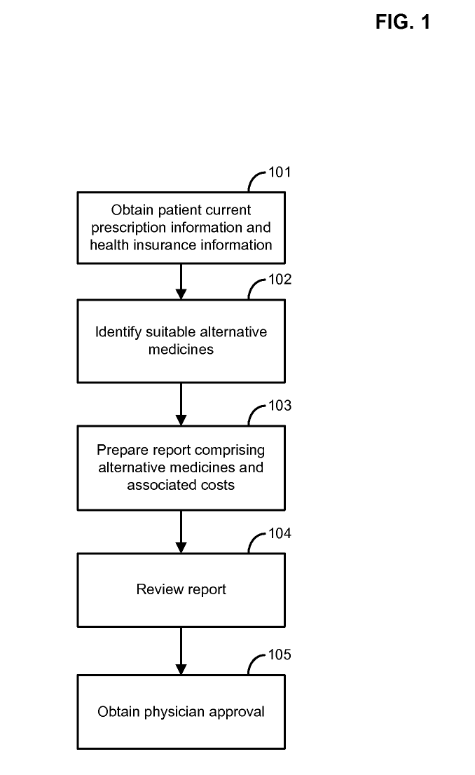

FIG. 1 is a block diagram of an exemplarily embodiment. Patient current prescription information and health insurance information can be obtained at block 101. That information can be reviewed in block 102 where suitable alternative prescriptions are identified along with cost information. A suitable alternative prescription can comprise a prescription that is generally used for treating the same medical condition(s) as the current prescription. A prescription can comprise a drug, dose, frequency, or combination thereof, that a physician approves for a patient. A suitable alternative prescription can be defined as an alternative prescription(s) identified that can be presented to the patient and/or the patient's physician as an option for replacement of a current prescription. The alternative prescription(s) may or may not be approved by the physician for use by the patient. More than one suitable alternative prescription may exist or no alternative prescription may exist for any given current prescription that would be medically appropriate for the given patient and their individual medical condition. The specific alternative prescription(s) can comprise a specific drug in combination with the dose and frequency the medication is taken. This review and identification can be automated in whole or in part. A report detailing the alternative drugs and their associated costs can be generated at block 103. In block 104 the report can be reviewed by the patient and the patient can select any alternative prescriptions the patient desires to obtain. In block 105, approval to fill the alternatives can be obtained from the prescribing physician.

The receipt of information from the patient can occur orally such as in person or over the telephone, via e-mail, via a computer interface on the World Wide Web, via a written communication or via other means of communications, including forms of electronic communications. The information can include combinations of the following: identifying information of the patient, prescribing physicians contact information, prescription information, insurance information, and medical history.

Identification of suitable alternative prescriptions can take place in a plurality of ways. The alternative prescriptions can be a different medication or the same medication with differing formulations or dosing regimes, or generic versions of a medication. Pharmaceutical information including various alternatives can reside in a publication or a computerized database whereby the input of a prescribed medication can result in a listing of possible alternative medications. Optionally, the database can comprise cost information for the various medications. Optionally, the database can comprise insurance payment information and/or plan formulary whereby the insurance benefits of the patient can be used to compute the actual cost to the patient after taking into account the patient's insurance benefits. Combinations of the foregoing information can also be included in the database.

The alternative prescriptions can be presented in written format. The review of the alternative prescriptions with the patient can take place over the telephone, in person, or via electronic means, including display on a web page on the World Wide Web. The review of alternative prescriptions can comprise the patient reviewing the list of alternative prescriptions and any accompanying information, if any. The review process can be complete when the patient selects alternative prescriptions or decides to remain with the current prescriptions. This selection can be oral or using a written format, such as marking the selected alternative. The selection can also be performed via electronic means, such as e-mail communications, facsimile, selection via a website on the World Wide Web, combinations thereof, and the like. Patient consent to contact the prescribing physician regarding alternative prescriptions for their medical condition can be obtained at any point in the process prior to obtaining consent from the prescribing physician. Consent can be written or via electronic means, including facsimile, electronic mail, or via a web page on the World Wide Web, combinations thereof, and the like.

Obtaining consent from the prescribing physician can be performed in ways known in the art, including orally, in writing, via electronic means, a combination thereof, and the like. Information presented to the physician at the time of a request for approval can comprise the current prescription and the alternative prescriptions.

The filling of the alternative prescription(s) can be performed in ways known in the art including dispensing the medicines and delivery via the postal service, couriers, in person, combinations thereof, and the like.

Optionally, additional information can be obtained from the patient such as allergy information. Optionally, information can be given to the patient in addition to the list of alternative prescriptions, such as which Medicare plans cover their prescriptions, where and how to qualify for free or reduced price drugs, and/or management assistance of their pharmaceutical benefits.

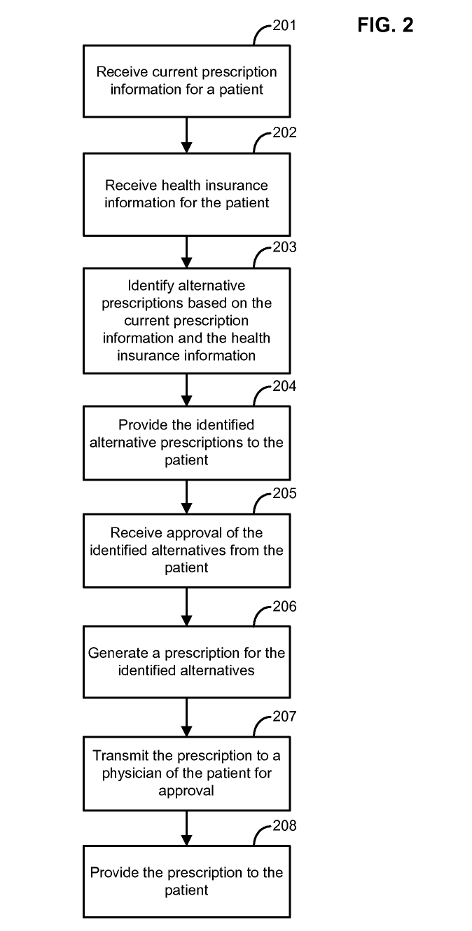

Provided, and illustrated in FIG. 2, are methods for providing an alternative prescription comprising receiving current prescription information for a patient wherein the current prescription information comprises a drug, a dosage for the drug, and a frequency for the drug at block 201, receiving health insurance information for the patient at block 202, identifying alternative prescriptions based on the current prescription information and the health insurance information at block 203, providing the identified alternative prescriptions to the patient at block 204, receiving approval of the identified alternatives from the patient at block 205, generating a prescription for the identified alternatives wherein the prescription comprises a current drug used and a specific drug change option at block 206, transmitting the prescription to a physician of the patient for approval at block 207, and providing the prescription to the patient at block 208.

The current prescription information can comprise a plurality of drugs currently prescribed to the patient, a dose associated with each of the plurality of drugs, a frequency associated with each of the plurality of drugs, and combinations thereof. The methods can further comprising filling the prescription for the patient.

The health insurance information can include, for example, an insurance company or health plan name or other identifier; insurance company or health plan contact information which can comprise address, telephone number(s), billing addresses; specific patient identifier (which can comprise alphanumeric characters); group identification; and any other information the insurance company might require for submission of claims or bills to the insurance company for payment by the insurance company on behalf of the insured or patient. Specifically for the coverage of prescription medications this information can comprise the name of the company or pharmacy benefits manager also known as PBM, the Rx Bin number, group plan identification, individual patient identification, PCN identification and the telephone number of the online processor for assistance in getting claims paid. An identifier can be an alphanumeric identifier unique to the patient, plan and/or group. There can be other information and/or specific patient identification information needed to bill a specific insurance company or health plan not required by other insurance companies. There can also be multiple plans that pay for a portion of the patients health care services such as hospitalization, doctor visits, prescription medications or other health services.

The step of identifying alternative prescriptions based on the current prescription information and the health insurance information can comprise determining an alternative drug for a current prescription, determining an alternative dosage for a current prescription, determining an alternative frequency for a current prescription, determining an alternative dosage and frequency for a current prescription, determining an alternative drug, dosage and frequency for a current prescription, determining an alternative drug and frequency for a current prescription, determining an alternative drug and dosage for a current prescription, determining a less expensive prescription option that achieves a similar health effect as a current prescription, and the like.

An alternative drug can be a different drug from a currently prescribed drug that is generally accepted by the medical profession to have a similar medical benefit for a patient with a given medical condition. A different drug is defined as any prescription drug that requires a new prescription from the physician to dispense the alternative drug. An alternative drug can be two separate drugs replacing a single drug that is made as a combination drug containing exactly the same two drugs identified as the alternative. An alternative drug can be the same chemical compound but it is made in a way such that the alternative drug is absorbed by the body at a different rate such as a slow or extended release medication compared to a standard rate of absorption. The alternative drug can be identified as a possible replacement or alternative drug because it has a very similar effect on a human body but it consists of a different chemical compound. The alternative drug can be a drug that is in the same class of drug as the currently prescribed drug. Other factors that can only be determined by the patient's physician can make any alternative drug(s) identified inappropriate as an alternative drug, these reasons include but are not limited to the specific patient's medical condition(s), allergies, general patient health, side effects of specific medications and even the individual physician's personal experience treating patients with various drugs.

An alternative dosage can be a different strength of a drug. For example 50 mg of drug X can be an equivalent dose as a 25 mg dose of drug Y. A prescription requires the dose of the drug to be identified in order to dispense the drug.

An alternative frequency can be a change in how often a drug is taken. For any specific drug to be effective it must be taken at regular intervals as prescribed by the physician. A prescription requires that a frequency be defined. For example a currently prescribed drug may be taken once daily and the alternative drug identified may be taken twice daily to achieve the same medical benefit as the originally prescribed drug. There may be no change in frequency because both the currently prescribed drug and any alternative drugs identified are taken with the same frequency.

The less expensive prescription option can be less expensive for at least one of the patient, an insurance company, an employer of the patient, and the like. The cost of medications is commonly shared by the patient and the insurance company or health plan paying medical benefits for a patient. The most common format is one in which the patient pays a co-pay or portion of the cost of the drug. A common method used is one in which a lower co-pay is required for generic drugs and a higher co-pay is required for brand name drugs. The patient can save money when the alternative medication replaces a brand name drug with a generic drug. For example if the co-pays are $10 for a generic drug and $25 for a brand name drug for a one months supply, then the savings of the use of the alternative drug if approved by the physician would be $15 month or $180 per year. The insurance company or health plan saves money when their portion of the expense of the alternative medication identified is lower than their portion of the cost of the originally prescribed medication. The specific alternatives identified by the program can be chosen to maximize the savings of one of these two parties or it can be made to assure cost savings for both parties.

The specific drug change option can comprise a specific drug, a dose, a frequency, an amount of monetary savings, a combination thereof, and the like. For example, the specific drug change option can indicate: an alternative drug, frequency, and dosage; an alternative dosage and frequency; and alternative drug and frequency; an alternative drug; an alternative dosage; an alternative frequency; and the like. The methods and systems can identify a specific savings that would be realized with each different option identified based on the specific health insurance information of the patient. This provides the patient with the specific information to judge if a particular option is worth any effort required to implement the change.

The step of transmitting the prescription to a physician of the patient for approval further can comprise permitting the physician to alter the prescription.

Embodiments can comprise a computer with a connection to the Internet, acting as a web server, wherein the methods described herein are implemented on the web server whereby the patient can access the computer implemented method so as to enter data, receive data, and review information pursuant to the methods described herein.

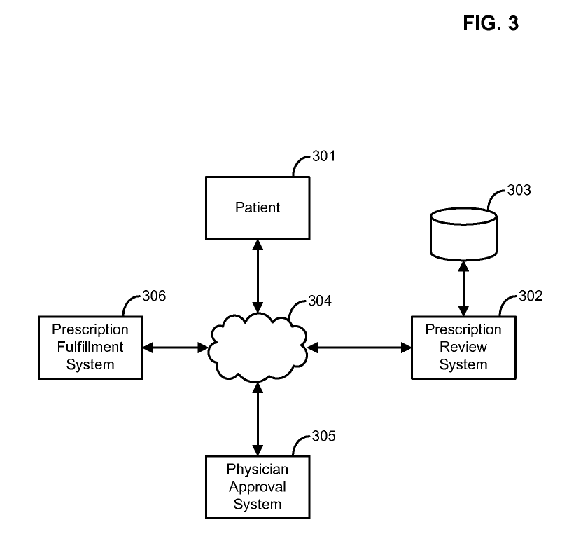

FIG. 3 illustrates generally, an exemplary system implementing the disclosed method. This exemplary system is only an example of a system and is not intended to suggest any limitation as to the scope of use or functionality of system architecture. Neither should the system be interpreted as having any dependency or requirement relating to any one or combination of components illustrated in the exemplary system. FIG. 3 illustrates the primary components and relationships of the disclosed system and method. A Patient 301 interacts with a Prescription Review System 302 via a network such as the Internet 308 through an internet-enabled computer connection. The Patient 301 can interact with the Prescription Review System 302 via an internet enabled personal computer running a standard web-browser. The Patient 301 can also interact with the Prescription Review System 302 via standard Point of Sale transactions. An example of such Point of Sale transactions includes conducting a transaction at a physical location such as a pharmacy, a health care facility, and the like. The Patient 301 is also capable of interacting with the Prescription Review System 302 through a plurality of internet enabled kiosks at various physical locations. The Prescription Review System 302 is capable of conducting a plurality of transactions with a plurality of Patients 301. Through the Prescription Review System 302, the Patient 301 is able to view a host website and upload a current prescription of the Patient 301. An exemplary current prescription can comprise a drug, a dosage for the drug, and a frequency of administration for the drug. The Prescription Review System 302 supports one or more prescription related databases 303. The prescription related databases 303 can comprise data relating to: drug equivalents, manufacturers and/or distributors costs of purchasing drugs, insurance plan formularies that identify what drugs are covered by each plan, drug efficacy studies, FDA related data, relative medical efficacy of different drugs in treating the same medical condition, medications that are in the same class and generation of drugs, drug options for expensive drugs, and other data that provides similar information. It will be readily apparent to those skilled in the art that the databases can be any suitable database management system such as Oracle, Informix, Sybase, SQL Server, Access, mySQL, PostgreSQL, or the like.

Once the Prescription Review System 302 receives the current prescription, the Prescription Review System 302 can identify alternative prescriptions for the Patient 301. The alternative prescriptions can be compiled into a prescription that can be transmitted to the Patient 301 for approval. The patient may choose to not present to their physician some of the options identified for a variety of reasons. These include, but are not limited to, the alternative medication is one that the patient already tried and was not effective or the patient had a specific side effect from one of the alternatives identified. Another reason a patient would not want to have an option presented to their physician is that the extra effort required, such as taking more pills a day, is not worth the cost savings received.

Once approved by the Patient 301, the prescription can be transmitted to a Physician Approval System 305. This transmission can take place through a network such as the Internet 304. The transmission can also take place via telephone, facsimile, email, postal mail, and the like. In another embodiment, the prescription can be transmitted to the Physician Approval System 305 and the Patient 301 simultaneously. In another embodiment, the prescription can be transmitted to the Physician Approval System 305 without Patient 301 approval.

Once received by the Physician Approval System 305, a physician can review the prescription and make any modifications necessary in light of the medical status of Patient 301 and approve the prescription. The approved prescription can be transmitted from the Physician Approval System 305 to the Patient 301. In another embodiment, the approved prescription can be transmitted from the Physician Approval System 305 to the Patient 201 and to a Prescription Fulfillment System 305. In another embodiment, the approved prescription can be transmitted from the Physician Approval System 305 only to a Prescription Fulfillment System 305. The transmissions can take place through a network such as the Internet 304. The transmissions can also take place via telephone, facsimile, email, postal mail, and the like.

The Prescription Fulfillment System 305 can be a pharmacy that is local to the Patient 301, enabling the Patient 301 to pick up the filled prescription. In other embodiments, Prescription Fulfillment System 305 can be an online pharmacy, whereby prescriptions are filled remotely from the Patient 301 and subsequently mailed to the Patient 301. The Prescription Review System 302 and the Prescription Fulfillment System 305 can implement electronic funds transfers between a multitude of financial accounts as is known to one skilled in the art.

Prescription Review System 302, Physician Approval System 305, and Prescription Fulfillment System 305 can be components of a single system, or alternatively can be components of a distributed system. For example, in an embodiment, Prescription Review System 302, Physician Approval System 305, and Prescription Fulfillment System 305 can all be in a computer system located in a single health care facility. In other embodiments, Prescription Review System 302 can be located remotely from any health care facility, Physician Approval System 305 can be located remotely from any health care facility, and Prescription Fulfillment System 305 can be located remotely from any health care facility. In another embodiment, Prescription Review System 302, Physician Approval System 305, and Prescription Fulfillment System 305 can be located on a single computing device, accessible via the Internet 304 from a remote location.

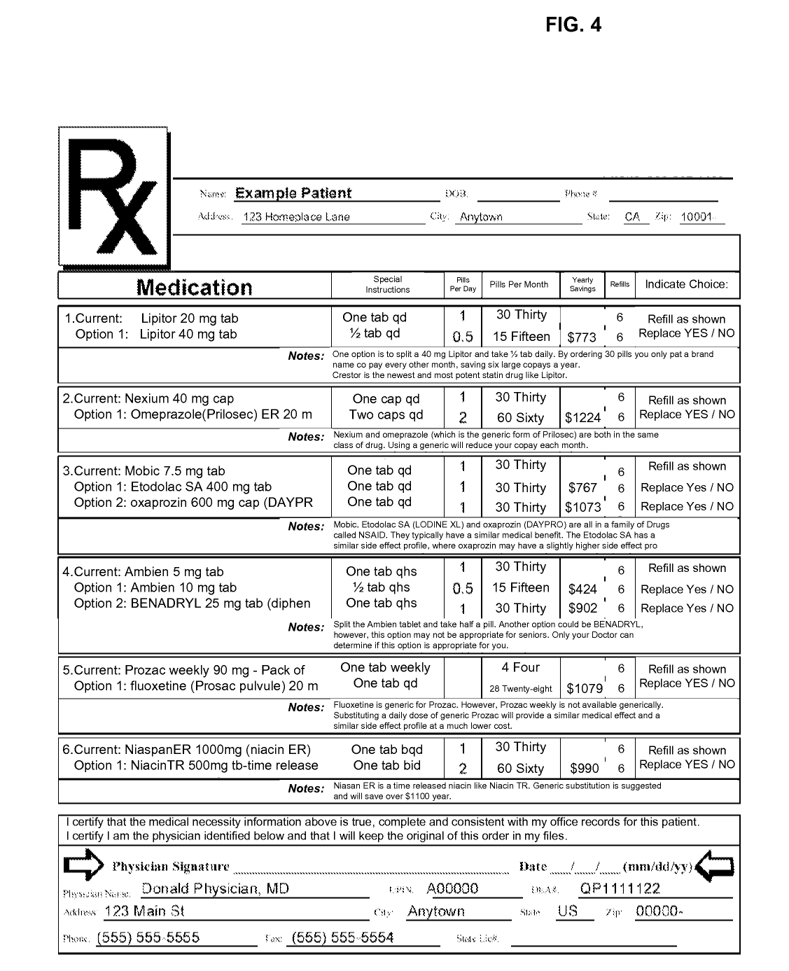

FIG. 4 illustrates an exemplary prescription that can be generated as a result of the methods and systems disclosed. The top portion of FIG. 4 identifies a specific patient by name, address, date of birth and phone number. The bottom portion of FIG. 4 identifies the patient's physician by name, address, phone number, fax number, DEA license number, UPIN number and provides a place to sign and date the prescription. The main body of FIG. 4 identifies the current prescriptions along with alternative prescription(s). Both prescriptions includes drug name, dose, special instructions, frequency, number of pills to dispense, cost savings and number of refills associated with each prescription. There is an area for each prescription for the physician to indicate “yes” or “no” to indicate if a particular alternative prescription is medically appropriate for the patient. Below each current prescription and alternative prescription(s) is a “Notes” area where additional information is provided that can assist the patient and/or physician in better understanding the alternative prescriptions identified.

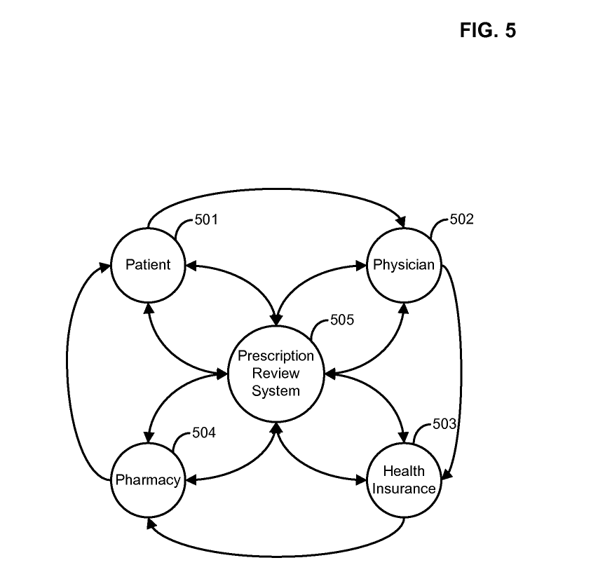

FIG. 5 describes examples of synergies generated by the disclosed methods and systems. The Patient 501 benefits through receiving drug cost savings and/or easier drug administration. This allows the Patient 501 to properly treat medical conditions that might otherwise go untreated because of high drug costs. The Physician 502 benefits through being enabled to more effectively treat the Patient 501, thus ensuring the Patient 501 can continue to utilize the services of Physician 502. Health Insurance 503 benefits through subsidize less on the drugs prescribed, thus ensuring affordable health insurance for the Patient 501. Pharmacy 504 benefits through receiving accurate prescriptions, allowing for more efficient and profitable prescription fulfillment. These benefits are conveyed by and through the Prescription Review System 505, serving as the medium through which all the aforementioned benefits flow.

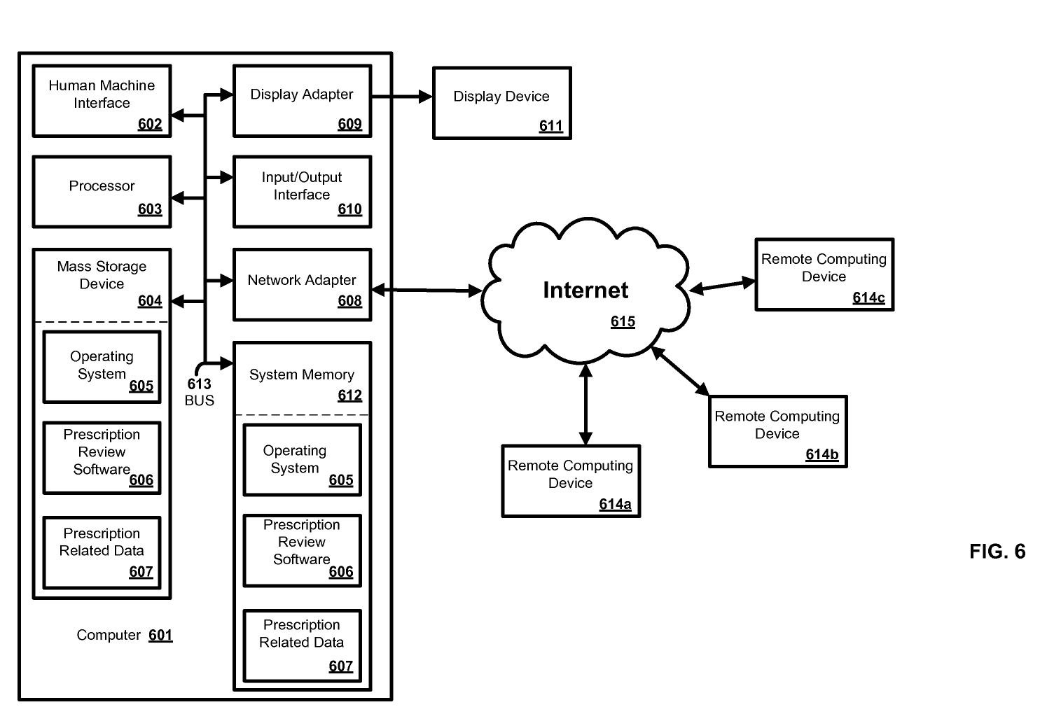

FIG. 6 is a block diagram illustrating an exemplary operating environment for performing the disclosed method. This exemplary operating environment is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment.

The present methods and systems can be operational with numerous other general purpose or special purpose computing system environments or configurations. Examples of well known computing systems, environments, and/or configurations that can be suitable for use with the system and method comprise, but are not limited to, personal computers, server computers, laptop devices, and multiprocessor systems. Additional examples comprise set top boxes, programmable consumer electronics, network PCs, minicomputers, mainframe computers, distributed computing environments that comprise any of the above systems or devices, and the like.

The processing of the disclosed methods and systems can be performed by software components. The disclosed system and method can be described in the general context of computer-executable instructions, such as program modules, being executed by one or more computers or other devices. Generally, program modules comprise computer code, routines, programs, objects, components, data structures, etc. that perform particular tasks or implement particular abstract data types. The disclosed method can also be practiced in grid-based and distributed computing environments where tasks are performed by remote processing devices that are linked through a communications network. In a distributed computing environment, program modules can be located in both local and remote computer storage media including memory storage devices.

Further, one skilled in the art will appreciate that the system and method disclosed herein can be implemented via a general-purpose computing device in the form of a computer 601. The components of the computer 601 can comprise, but are not limited to, one or more processors or processing units 603, a system memory 612, and a system bus 613 that couples various system components including the processor 603 to the system memory 612.

The system bus 613 represents one or more of several possible types of bus structures, including a memory bus or memory controller, a peripheral bus, an accelerated graphics port, and a processor or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI) bus also known as a Mezzanine bus. The bus 613, and all buses specified in this description can also be implemented over a wired or wireless network connection and each of the subsystems, including the processor 603, a mass storage device 604, an operating system 605, prescription review software 606, prescription related data 607, a network adapter 608, system memory 612, an Input/Output Interface 610, a display adapter 609, a display device 611, and a human machine interface 602, can be contained within one or more remote computing devices 614a,b,c at physically separate locations, connected through buses of this form, in effect implementing a fully distributed system.

The computer 601 typically comprises a variety of computer readable media. Exemplary readable media can be any available media that is accessible by the computer 601 and comprises, for example and not meant to be limiting, both volatile and non-volatile media, removable and non-removable media. The system memory 612 comprises computer readable media in the form of volatile memory, such as random access memory (RAM), and/or non-volatile memory, such as read only memory (ROM). The system memory 612 typically contains data such as prescription related data 607 and/or program modules such as operating system 605 and prescription review software 606 that are immediately accessible to and/or are presently operated on by the processing unit 603.

In another aspect, the computer 601 can also comprise other removable/non-removable, volatile/non-volatile computer storage media. By way of example, FIG. 6 illustrates a mass storage device 604 which can provide non-volatile storage of computer code, computer readable instructions, data structures, program modules, and other data for the computer 601. For example and not meant to be limiting, a mass storage device 604 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 604, including by way of example, an operating system 605 and prescription review software 606. Each of the operating system 605 and prescription review software 606 (or some combination thereof) can comprise elements of the programming and the prescription review software 606. Prescription related data 607 can also be stored on the mass storage device 604. Prescription related data 607 can be stored in any of one or more databases known in the art. Examples of such databases comprise, DB2®, Microsoft® Access, Microsoft® SQL Server, Oracle®, mySQL, PostgreSQL, and the like. The databases can be centralized or distributed across multiple systems.

In another aspect, the user can enter commands and information into the computer 601 via an input device (not shown). Examples of such input devices comprise, but are not limited to, a keyboard, pointing device (e.g., a “mouse”), a microphone, a joystick, a scanner, tactile input devices such as gloves, and other body coverings, and the like These and other input devices can be connected to the processing unit 603 via a human machine interface 602 that is coupled to the system bus 613, but can be connected by other interface and bus structures, such as a parallel port, game port, an IEEE 1394 Port (also known as a Firewire port), a serial port, or a universal serial bus (USB).

In yet another aspect, a display device 611 can also be connected to the system bus 613 via an interface, such as a display adapter 609. It is contemplated that the computer 601 can have more than one display adapter 609 and the computer 601 can have more than one display device 611. For example, a display device can be a monitor, an LCD (Liquid Crystal Display), or a projector. In addition to the display device 611, other output peripheral devices can comprise components such as speakers (not shown) and a printer (not shown) which can be connected to the computer 601 via Input/Output Interface 610.

The computer 601 can operate in a networked environment using logical connections to one or more remote computing devices 614a,b,c. By way of example, a remote computing device can be a personal computer, portable computer, a server, a router, a network computer, a peer device or other common network node, and so on. Logical connections between the computer 601 and a remote computing device 614a,b,c can be made via a local area network (LAN) and a general wide area network (WAN). Such network connections can be through a network adapter 608. A network adapter 608 can be implemented in both wired and wireless environments. Such networking environments are conventional and commonplace in offices, enterprise-wide computer networks, intranets, and the Internet 615.

For purposes of illustration, application programs and other executable program components such as the operating system 605 are illustrated herein as discrete blocks, although it is recognized that such programs and components reside at various times in different storage components of the computing device 601, and are executed by the data processor(s) of the computer. An implementation of prescription review software 606 can be stored on or transmitted across some form of computer readable media. Computer readable media can be any available media that can be accessed by a computer. By way of example and not meant to be limiting, computer readable media can comprise “computer storage media” and “communications media.” “Computer storage media” comprise volatile and non-volatile, removable and non-removable media implemented in any method or technology for storage of information such as computer readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

The methods and systems can employ Artificial Intelligence techniques such as machine learning and iterative learning. Examples of such techniques include, but are not limited to, expert systems, case based reasoning, Bayesian networks, behavior based AI, neural networks, fuzzy systems, evolutionary computation (e.g. genetic algorithms), swarm intelligence (e.g. ant algorithms), and hybrid intelligent systems (e.g. Expert inference rules generated through a neural network or production rules from statistical learning).

The present methods can be implemented in a typical “e-commerce” environment, in other words, the conducting of business communication and transactions over networks and through computers; the buying and selling of goods and services, and the transfer of funds, through digital communications. However, the methods can also be implemented in traditional point-of-sale environments.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

1. A method for providing an alternative prescription comprising:
receiving current prescription information for a patient wherein the current prescription information comprises a drug, a dosage for the drug, and a frequency for the drug;
receiving health insurance information for the patient;
identifying alternative prescriptions based on the current prescription information and the health insurance information;
providing the identified alternative prescriptions to the patient;
receiving approval of the identified alternatives from the patient;
generating a prescription for the identified alternatives wherein the prescription comprises a current drug used and a specific drug change option;
transmitting the prescription to a physician of the patient for approval; and
providing the prescription to the patient.

2. The method of claim 1, wherein the current prescription information comprises a plurality of drugs currently prescribed to the patient.

3. The method of claim 1, wherein identifying alternative prescriptions based on the current prescription information and the health insurance information comprises determining an alternative drug for a current prescription.

4. The method of claim 1, wherein identifying alternative prescriptions based on the current prescription information and the health insurance information comprises determining an alternative dosage for a current prescription.

5. The method of claim 1, wherein identifying alternative prescriptions based on the current prescription information and the health insurance information comprises determining an alternative frequency for a current prescription.

6. The method of claim 1, wherein identifying alternative prescriptions based on the current prescription information and the health insurance information comprises determining an alternative dosage and frequency for a current prescription.

7. The method of claim 1, wherein identifying alternative prescriptions based on the current prescription information and the health insurance information comprises determining an alternative drug, dosage and frequency for a current prescription.

8. The method of claim 1, wherein identifying alternative prescriptions based on the current prescription information and the health insurance information comprises determining an alternative drug and frequency for a current prescription.

9. The method of claim 1, wherein identifying alternative prescriptions based on the current prescription information and the health insurance information comprises determining an alternative drug and dosage for a current prescription.

10. The method of claim 1, wherein identifying alternative prescriptions based on the current prescription information and the health insurance information comprises determining a less expensive prescription option that achieves a similar health effect as a current prescription.

11. The method of claim 10, wherein the less expensive prescription option is less expensive for at least one of the patient, an insurance company, and an employer of the patient.

12. The method of claim 1, wherein the specific drug change option comprises a specific drug, a dose, a frequency, and an amount of monetary savings.

13. The method of claim 1, wherein transmitting the prescription to a physician of the patient for approval further comprises permitting the physician to alter the prescription.

14. The method of claim 1, further comprising filling the prescription for the patient.

15. A system for providing an alternative prescription comprising:
a memory configured for storing data related to a prescription;
a processor, coupled to the memory, wherein the processor is configured for performing the steps comprising:
receiving current prescription information for a patient wherein the current prescription information comprises a drug, a dosage for the drug, and a frequency for the drug;
receiving health insurance information for the patient;
identifying alternative prescriptions based on the current prescription information and the health insurance information;
providing the identified alternative prescriptions to the patient;
receiving approval of the identified alternatives from the patient;
generating a prescription for the identified alternatives wherein the prescription comprises a current drug used and a specific drug change option;
transmitting the prescription to a physician of the patient for approval; and
providing the prescription to the patient.

16. The system of claim 15, wherein the current prescription information comprises a plurality of drugs currently prescribed to the patient.

17. The system of claim 15, wherein identifying alternative prescriptions based on the current prescription information and the health insurance information comprises determining an alternative drug, dosage and frequency for a current prescription.

18. The system of claim 15, wherein the specific drug change option comprises a specific drug, a dose, a frequency, and an amount of monetary savings

19. The system of claim 15, wherein transmitting the prescription to a physician of the patient for approval further comprises permitting the physician to alter the prescription.

20. A computer readable medium with computer executable instructions embodied thereon for providing an alternative prescription comprising:
receiving current prescription information for a patient wherein the current prescription information comprises a drug, a dosage for the drug, and a frequency for the drug;
receiving health insurance information for the patient;
identifying alternative prescriptions based on the current prescription information and the health insurance information;
providing the identified alternative prescriptions to the patient;
receiving approval of the identified alternatives from the patient;
generating a prescription for the identified alternatives wherein the prescription comprises a current drug used and a specific drug change option;
transmitting the prescription to a physician of the patient for approval; and
providing the prescription to the patient.
